(19) United States
(12) Patent Application Publication
(10) Pub. No.: US 2011/0281788 A1
(43) Pub. Date: Nov. 17, 2011

(54) ANTI FUNGAL THERAPY

(76) Inventors: Peter John Coote, St. Andrews (GB); Mark Harris, St. Andrews (GB)

(21) Appl. No.: 13/119,806

(22) PCT Filed: Sep. 17, 2009
(86) PCT No.: PCT/GB2009/002226

§ 371 Date: Aug. 1, 2011

(30) Foreign Application Priority Data
Sep. 18, 2008 (GB) 0817121.7

(51) Int. Cl. A61K 38/12; A61P 31/10

(52) U.S. Cl. 514/3.4; 514/3.6

(57) ABSTRACT

The present invention relates to the treatment of fungal infections and provides compounds, compositions, methods and for treating the same.

### FIELD OF THE INVENTION

The present invention relates to the treatment of fungal infections and provides compounds, compositions and methods for treating the same.

### BACKGROUND OF THE INVENTION

The World Health Organisation has stated that in the modern world infectious disease still accounts for over 20% of all human morbidity and mortality. A recent UK-Government Foresight document (Infectious Diseases: preparing for the future) identified eight future disease categories that will become particularly important in the next 10-25 years, one of which is the emergence of multiply drug-resistant strains of human pathogens.

In recent years there has been a significant increase in the number of human fungal infections (nearly a 5-fold increase in candidaemia). In the USA, Candida albicans is the fourth leading cause of vascular catheter-related infections and the third leading cause of urinary catheter-related infection. Similarly, Candida species rank fifth among causes of nosocomial bloodstream infections. Also, the incidence of invasive aspergillosis has increased 4-fold over a decade in Europe and in some groups, such as those undergoing allogeneic bone marrow transplantation; mortality is in excess of 90%. The rise in fungal infections has occurred because of increased patient exposure to broad-spectrum antibiotics resulting in changes to the normal host flora, cancer chemotherapy, and organ and bone marrow transplantation.

Importantly, many of the existing antifungal drugs have undesirable side effects, are ineffective against new or re-emerging fungi and may promote the development of resistance in patients undergoing treatment. In addition, there are far fewer antifungal drugs than, for example, antibacterial drugs. As such there is a need to develop new drugs and/or novel routes/regimes to target fungal pathogens.

The echinocandins are a relatively new class of antifungals that inhibit the synthesis of β-D-glucan in fungal cell walls and are fungicidal. They are synthetically modified lipopeptides, with poor oral absorption and are thus administered intravenously. While, echinocandin antifungal agents have been found to have less side affects than, for example, polyene antifungal agents such as amphotericin B, adverse effects have been reported and include headache, fever, liver toxicity, phlebitis, histamine release, haemolysis and rash.

In view of the above, the shortage of new antifungal agents and problems associated with existing compounds creates a need for alternative methods of treating fungal infections.

### SUMMARY OF INVENTION

The present invention is based on the finding that the use of a combination of compounds for treating fungal infections provides a more efficacious and potent means of treatment. In particular, the inventors have discovered that a combined or combinatorial approach using, for example, an echinocandin and an antimicrobial peptide, provides a more effective means of treatment.

Accordingly and in a first aspect, the present invention provides an echinocandin and an antimicrobial peptide, for use in treating a fungal infection. In one embodiment, the invention provides a composition comprising an echinocandin and an antimicrobial peptide, for use in treating a fungal infection.

In a second aspect, the present invention provides the use of an echinocandin and an antimicrobial peptide, for the manufacture of a medicament for treating a fungal infection.

In view of the above, a third aspect of this invention provides a method of treating a subject suffering from a fungal infection, said method comprising the steps of treating the subject with a therapeutically effective amount of an echinocandin and an antimicrobial peptide.

As stated, the present invention concerns a combined/combinatorial approach to the treatment of fungal infections and provides compounds, compositions, medicaments and methods for use in treating fungal infections. The inventors have established that the problems associated with the use of echinocandins (for example, toxicity, side-effects, cost, ineffectiveness and/or the development of resistance) may be overcome by combining the use of one or more echinocandin compounds with one or more antimicrobial peptides. When compared to monotherapeutic approaches, particularly monotherapeutic treatments involving the use of echinocandin compounds or antimicrobial peptides, the combinatorial approach described herein provides a composition, medicament and/or treatment having greater efficacy and potency, fewer side effects, lower toxicity (i.e. safer) and a broader spectrum of activity. In addition, the inventors have determined that resistance to an antifungal treatment comprising a combination of any of the echinocandin compounds described herein with one or more antimicrobial peptides, is less likely to develop. A further advantage of the present invention is that by using lower quantities of each of the individual compounds (i.e. an echinocandin or antimicrobial peptide), the combinatorial approach to treating fungal infections described herein, may cost significantly less than other (for example, monotherapeutic) approaches.

In other words, when treating fungal infections, a composition, medicament or treatment comprising a combination of echinocandin compounds and antimicrobial peptides, exhibits a synergistic effect whereby, for example, the efficacy and potency of the combination is greater than the efficacy and potency of each of the components (i.e. the echinocandin compound or antimicrobial peptide) of the combination when used individually (i.e. in a monotherapeutic treatment regime).

One of skill in the art will be familiar with the antifungal compounds known collectively as the “echinocandins”. These compounds are synthetically modified lipopeptides which inhibit the synthesis of (1,3)-βD-glucan, an integral component of the fungal cell wall, resulting in cell wall damage and ultimately cell death. Accordingly, any of the antifungal compounds within the “echinocandin” class may be considered as potentially useful in the present invention.

The term “echinocandin” may also be taken to relate to specific compounds and in this regard, the compounds Echinocandin B, Cilofungin, Caspofungin (1-[(4R,5S)-5[(2-aminoethyl)amino]-N²-(10,12-dimethyl-1-oxotetradecyl)-4-hydroxy-L-ornithine]-5-[(3R)-3-hydroxy-L-ornithine]pneumocandin B₀), Micafungin and/or Anidulafungin may be considered as potentially useful echinocandins. One of skill in this field will appreciate that this is not an exhaustive list of echinocandin compounds and others may be used. Furthermore, it should be understood that derivatives and/or analogues/variants of any echinocandin compound, including those mentioned herein, may also be useful.

The term “fungal infection” should be understood to include any disease or condition the symptoms of which are caused or contributed to, by a fungus. In this regard, fungal infections may otherwise be known as “mycoses” and the invention may be taken to relate to the treatment of cutaneous, subcutaneous and/or systemic mycoses. Furthermore, fungal infections—and particularly those referred to as systemic mycoses, may be caused by pathogenic and/or opportunistic fungi.

Accordingly, in one embodiment, the present invention provides compounds, compositions, methods and medicaments for use in treating fungal infections (or mycoses) involving, for example, Epidermophyton, Microsporum, Trichophyton, Actinomadura, Cladosporium, Madurella, Phialophora, Sporothrix, Blastomyces, Coccidioides, Histoplasma, Paracoccidioides, Absidia, Candida, Aspergillus, Crytopcoccus, pneumocystis, Rizomucor, Rhizopus species. One of skill will appreciate that there may be other types of fungi not listed here, but which also cause infection and which may be treated using any of the compounds, compositions, methods and/or medicaments described herein.

The term “antimicrobial peptide” should be taken to encompass any peptide exhibiting antimicrobial or antibiotic activity. One of skill will appreciate that antimicrobial peptides may exhibit antiviral, antifungal and/or antibacterial activities and in a preferred embodiment, the antimicrobial peptides useful in the instant invention may be antifungal.

Antimicrobial peptides, while somewhat heterogeneous in length, sequence and structure, are typically small and those useful in the present invention may comprise 5-50 amino acids, preferably 10-40 amino acids and even more preferably 16-30 amino acids in length.

Despite their heterogeneous primary, secondary and/or tertiary structural features, the antimicrobial peptides for use in this invention are generally linear, α-helical antimicrobial peptides having an amphipathic structure. Nevertheless, other antimicrobial peptides such as, for example, natural and/or synthetic cyclic antimicrobial peptides, lipopeptides and lantibiotics are within the scope of this invention.

One of skill in this field will readily understand that amphipathic molecules and in particular amphipathic peptides, have hydrophobic domains comprising non-polar amino acid residues and hydrophilic domains comprising polar, positively charged residues. Accordingly, the antimicrobial peptides useful in this invention may include those classified as cationic peptides.

The term “lipopeptide” may be taken to encompass antimicrobial compounds such as, for example, the polymyxins including polymyxin B, E and/or M.

The term “lantibiotic” may relate to antimicrobial peptides containing polycyclic thioether amino acids as well as the unsaturated amino acids dehydroalanine and 2-aminoisobutyric acid. Both the long flexible chain-Type A and the globular type B lantibiotics may be encompassed within the term lantibiotic. For example the invention may relate to the use of the lantibiotic nisin.

In one embodiment, the compounds, compositions, medicaments and/or methods provided by this invention involve the use of one or more antimicrobial peptides selected from the group consisting of ranalexin, dermaseptin S3(1-16), those of the magainin family such as, for example, magainin 2, 6752, GS14K4, nisin, polymyxin B and Colistin sulfate (polymyxin E). It should be understood that while the aforementioned peptides represent examples of those suitable for use in the present invention, other antimicrobial or cationic/amphipathic peptides might also be useful. Moreover, it should be understood that the present invention should not be construed as being limited to the use of naturally occurring antimicrobial peptides and that synthetic antimicrobial peptides, synthetic versions or synthetic and/or naturally occurring mutants, homologues and/or analogues of any of those mentioned herein, may also be used.

Antimicrobial peptides of the magainin family comprise 23-34 amino acids. The specific peptide, magainin 2, comprises 23 amino acids and has the following primary structure:

GIGKFLHSAKKFGKAFVGEIMNS

The antimicrobial peptide ranalexin comprises 20 amino acids and has the following primary structure:

FLGGLIKIVPAMICAVTKKC

The antimicrobial peptide dermaseptin S3(1-16) is a truncated version of the full length dermaseptin S3 molecule (30 amino acids) and comprises the first 16 amino acids. Dermaseptis S3(1-16) has the following primary structure:

ALWKNMLKGIGKLAGK

The antimicrobial peptide 6752 is a cyclic peptide comprising 8 amino acids and having the following primary structure

S W F K T K S K

The antimicrobial peptide GS14K4 is a cationic peptide comprising 14 amino acids and having the following primary structure

V K Ld K Vd Y P L K V K Ld Y P

The lantibiotics potentially useful in this invention are described in the Eur. J. Biochem. 230 p 827-853 (1995). Nisin, for example, has the following primary structure:

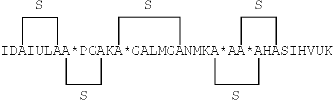

where “U” is dehydroalanine, “AsA” is lanthionine and “A*sA” is β methylanthionine.

Polymyxin B/E are cyclic peptides having long hydrophobic tails. The structure of these components are well known to one of skill in this field.

In view of the above, one embodiment of the present invention provides an echinocandin and an antimicrobial peptide selected from the group consisting of:

ranalexin;

(ii) magainin 2;

(iii) dermaseptin S3(1-16);

(iv) 6752;

(v) GS14K4;

(vi) Polymyxin B;

(vii) Colistin sulfate; and

(viii) nisin

for use in treating a fungal infection.

In a further embodiment, the present invention may provide one or more echinocandin compounds combined with one or more antimicrobial peptides, potentially selected from the group listed as (i)-(iii) above, for use in treating a fungal infection.

In a fourth aspect, the present invention provides a pharmaceutical composition comprising an echinocandin and an antimicrobial peptide for use in treating a fungal infection, in association with a pharmaceutically acceptable excipient, carrier or diluent.

Accordingly, the compounds provided by this invention may be formulated as pharmaceutical compositions (preferably sterile pharmaceutical compositions) comprising a pharmaceutically acceptable carrier or excipient. Such carriers or excipients are well known to one of skill in the art and may include, for example, water, saline, phosphate buffered saline, dextrose, glycerol, ethanol, ion exchangers, alumina, aluminium stearate, lecithin, serum proteins, such as serum albumin, buffer substances such as phosphates, glycine, sorbic acid, potassium sorbate, partial glyceride mixtures of saturated vegetable fatty acids, water salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polypropylene-block polymers, polyethylene glycol and wool fat and the like, or combinations thereof.

Pharmaceutical formulations include those suitable for oral, topical (including dermal, buccal and sublingual), rectal or parenteral (including subcutaneous, intradermal, intramuscular and intravenous), transdermal, nasal and pulmonary (for example by inhalation) administration. The formulation may, where appropriate, be conveniently presented in discrete dosage units and may be prepared by any of the methods well known in the art of pharmacy. Methods typically include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release-controlling matrix, or is coated with a suitable release-controlling film. Such formulations may be particularly convenient for prophylactic use. Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active compound in aqueous or oleaginous vehicles.

Injectable preparations may be adapted for bolus injection or continuous infusion. Such preparations are conveniently presented in unit dose or multi-dose containers, which are sealed after introduction of the formulation until required for use. Alternatively, an active compound may be in powder form that is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

It should be understood that in addition to the aforementioned carrier ingredients the pharmaceutical formulations described above may include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutically acceptable carriers are well known to those skilled in the art and include, but are not limited to, 0.1M and preferably 0.05 M phosphate buffer or 0.8% saline. Additionally, pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments.

Liquid or powder formulations may also be provided which can be sprayed or sprinkled directly onto the site to be treated, e.g. a wound or ulcer. Alternatively, a carrier such as a bandage, gauze, mesh or the like can be impregnated, sprayed or sprinkled with the formulation and then applied to the site to be treated.

Therapeutic formulations for veterinary use may conveniently be in either powder or liquid concentrate form. In accordance with standard veterinary formulation practice, conventional water-soluble excipients, such as lactose or sucrose, may be incorporated in the powders to improve their physical properties. Thus particularly suitable powders of this invention comprise 50 to 100% w/w and preferably 60 to 80% w/w of the active ingredient(s) and 0 to 50% w/w and preferably 20 to 40% w/w of conventional veterinary excipients. These powders may either be added to animal feedstuffs, for example by way of an intermediate premix, or diluted in animal drinking water.

Liquid concentrates of this invention suitably contain the compound or a derivative or salt thereof and may optionally include an acceptable water-miscible solvent for veterinary use, for example polyethylene glycol, propylene glycol, glycerol, glycerol formal or such a solvent mixed with up to 30% v/v of ethanol. The liquid concentrates may be administered to the drinking water of animals.

In general, a suitable dose of the one or more compounds of the invention may be in the range of about 1 μg to about 5000 μg/kg body weight of the subject per day, e.g., 1, 5, 10, 25, 50, 100, 250, 1000, 2500 or 5000 μg/kg per day. Where the compound(s) is a salt, solvate, prodrug or the like, the amount administered may be calculated on the basis the parent compound and so the actual weight to be used may be increased proportionately.

The compositions provided by this invention may comprise an echinocandin and an antimicrobial peptide in a single preparation/formulation such that they are administered to a subject together and at the same time. Alternatively, the medicament or treatment may comprise two or more different preparations/formulations each containing either one or more echinocandins compounds and/or one or more antimicrobial peptides. In this way, the echinocandins compound may be administered together with and at the same time as, an antimicrobial peptide or, alternatively, separately from (i.e. before or after) the antimicrobial peptide.

In addition to the above, one of skill in the art will appreciate that where the treatment of a fungal infection comprises the administration or use of one or more different compositions each containing one or more echinocandins compounds and/or one or more antimicrobial peptides, it may be possible to administer the various compositions via different routes. For example, the echinocandins compound may be administered topically and/or orally concurrently with or separately from an antimicrobial peptide which may be administered topically and/or parenterally.

As stated, the pharmaceutical compositions of this invention may be formulated for topical administration and as such may be presented as an ointment, solution or a suspension in an aqueous or non-aqueous liquid, or as an oil-in-water liquid emulsion. Such formulations may be particularly useful where the fungal infection affects, for example, the skin.

It may be possible to administer the echinocandin compounds and antimicrobial peptides described above transdermally, via some form of transdermal delivery device. Transdermal administration may be achieved with the use of impregnated coverings dressings, bandages or the like or via the use of some form of transdermal delivery device. Such devices are advantageous, particularly for the administration of a compound or compounds useful in the treatment of a cutaneous disease, as they may allow a prolonged period of treatment relative to, for example, an oral or intravenous medicament.

Examples of transdermal delivery devices may include, for example, a patch, dressing, bandage or plaster adapted to release a compound or substance through the skin of a patient. A person of skill in the art would be familiar with the materials and techniques which may be used to transdermally deliver a compound or substance and exemplary transdermal delivery devices are provided by GB2185187, U.S. Pat. No. 3,249,109, U.S. Pat. No. 3,598,122, U.S. Pat. No. 4,144,317, U.S. Pat. No. 4,262,003 and U.S. Pat. No. 4,307,717.

By way of example, an echinocandin compound and antimicrobial peptide for use in the present invention may be combined with some form of matrix or substrate, such as a non-aqueous polymeric carrier, to render it suitable for use in a transdermal delivery system. The echinocandin compound and antimicrobial peptide/matrix or substrate mixture may be further strengthened by the use of, for example, a woven or knit, non-woven, relatively open mesh fabric, to produce a patch, bandage, plaster or the like which may be releasably attached to a particular region of a patient's body. In this way, while in contact with a patient's skin, the transdermal delivery device releases the echinocandin compound and antimicrobial peptide through or to the skin.

### DETAILED DESCRIPTION

The present invention will now be described in detail with reference to the following figures which show:

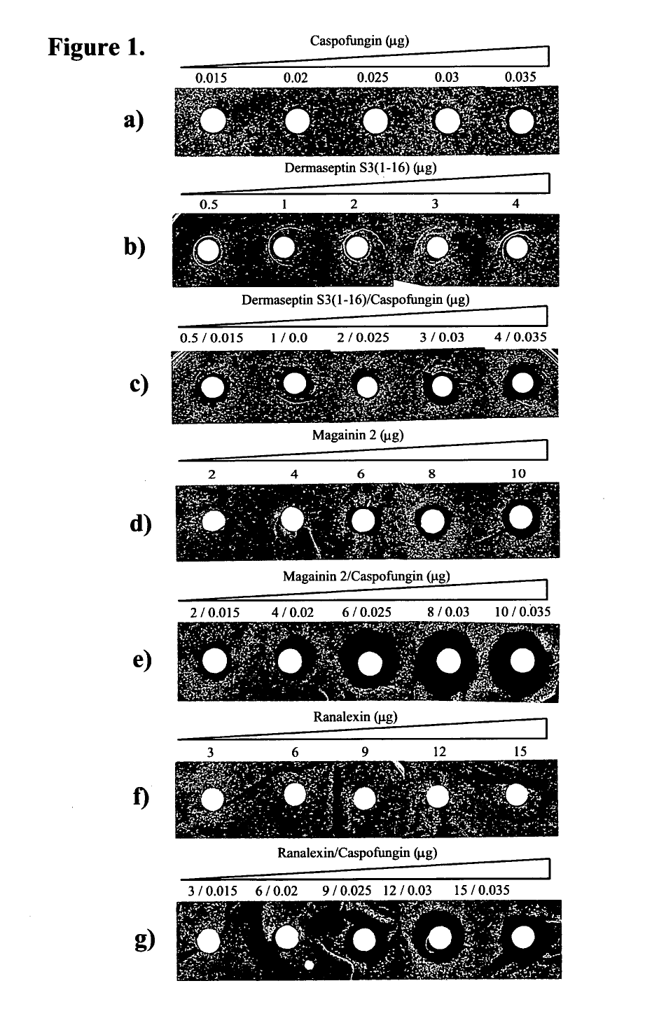

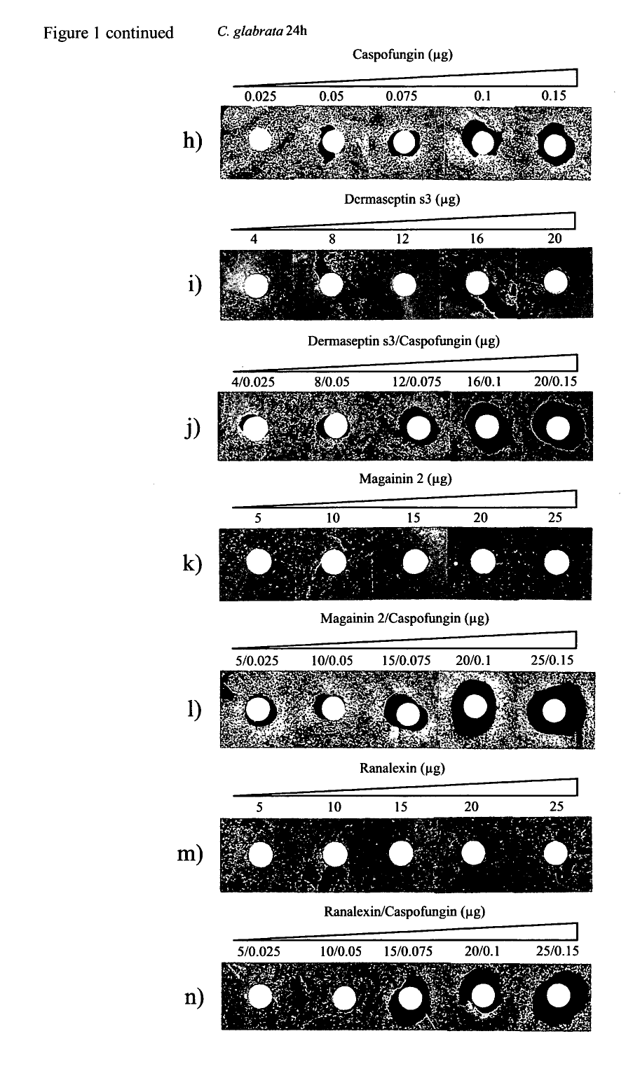

FIG. 1: shows the effect of antifungals impregnated into filter paper discs on the growth of yeast (Saccharomyces cerevisiae) on the surface of agar plates. Panel B shows the effect of caspofungin alone (no zones of clearing at the concentrations displayed). Panel A shows the effect of the antimicrobial peptide derma septin S3 (1-16) alone (again, no zones of clearing visible at the concentrations tested). Panel C shows the effect when caspofungin and dermaseptin S3(1-16) are applied together. Evident zones of clearing are visible. Using other antimicrobial peptides, Panel D shows the effect of magainin 2 alone and Panel F shows the effect of ranalexin alone. Both these peptides at the concentrations shown have minimal effect on yeast growth. However, when the magainin 2 and ranalexin are combined with caspofungin (Panels E and G) very clear zones of clearing become visible. The data shown in FIG. 1(h)-(n) are for the highly resistant yeast Candida glabrata which is difficult to treat due to its high intrinsic resistance to traditional antifungals. The data shown in FIGS. 1(o)-(u) are for Candida albicans. As above, the panels (h) through (u) show the effect of an echinocandin compound, an antimicrobial peptide and a combination of the two on Candida glabrata and Candida albicans.

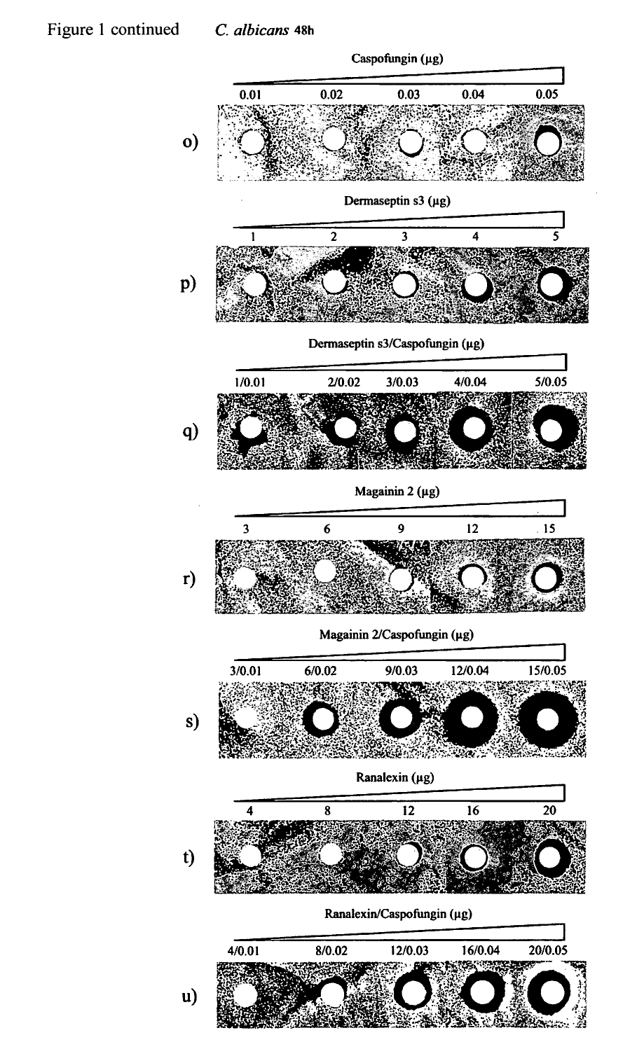

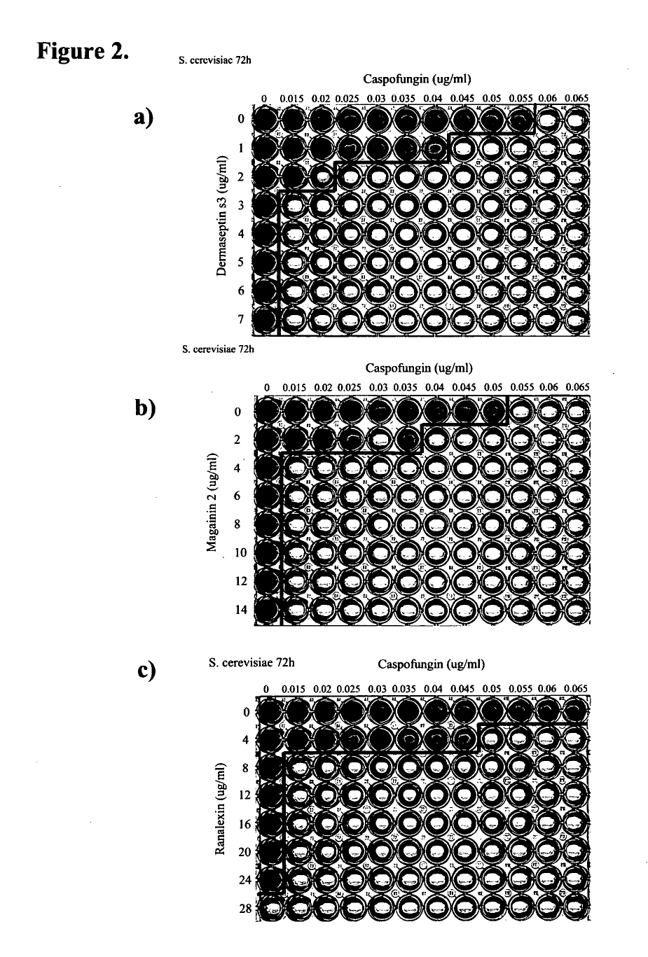

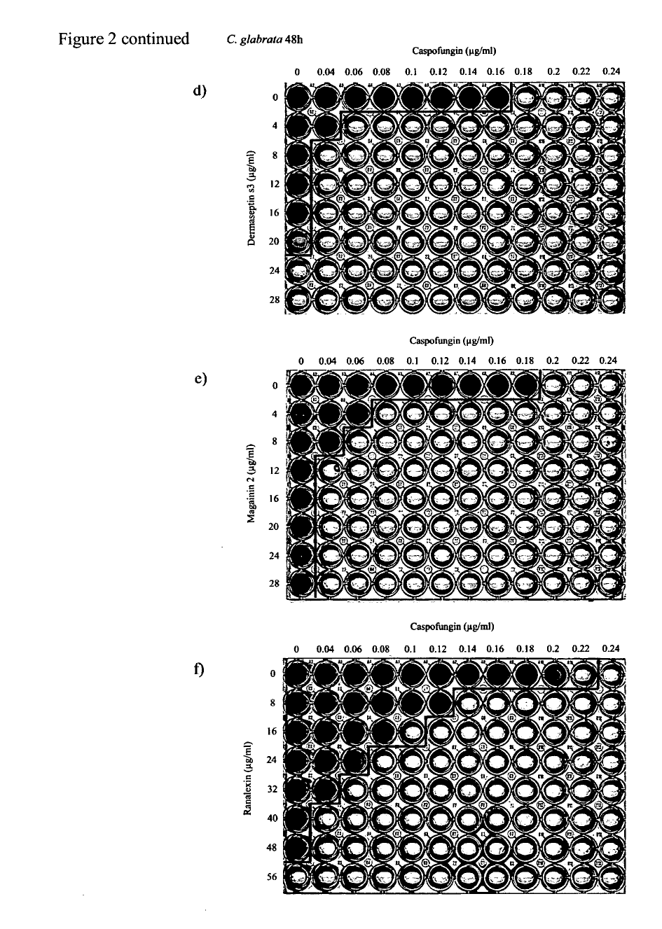

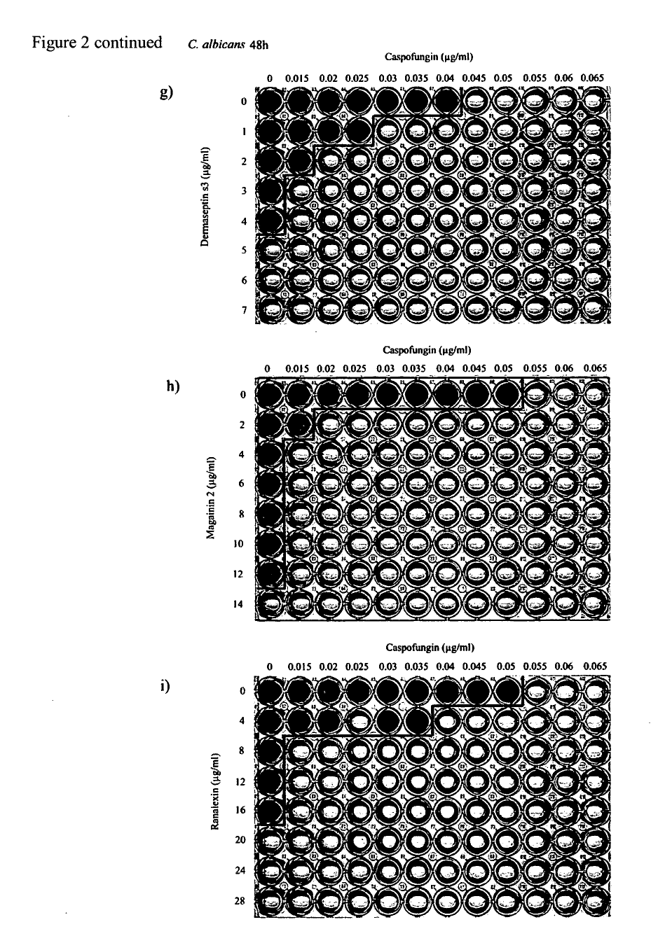

FIG. 2: shows the effect of combining antimicrobial peptides with caspofungin on yeast growth in liquid broth in the wells of a microtitre plate. Growth is indicated by the dark wells and no growth is indicated by the clear wells. Panel A shows the effect of combining caspofungin with dermaseptin S3(1-16). Panel B shows the effect of combining caspofungin with magainin 2. Panel C shows the effect of combining caspofungin with ranalexin. Panels D through I show the effect of the same echinocandin/antimicrobial peptide combinations on Candida glabrata (panels D, E and F) and Candida albicans (panels G, H and I).

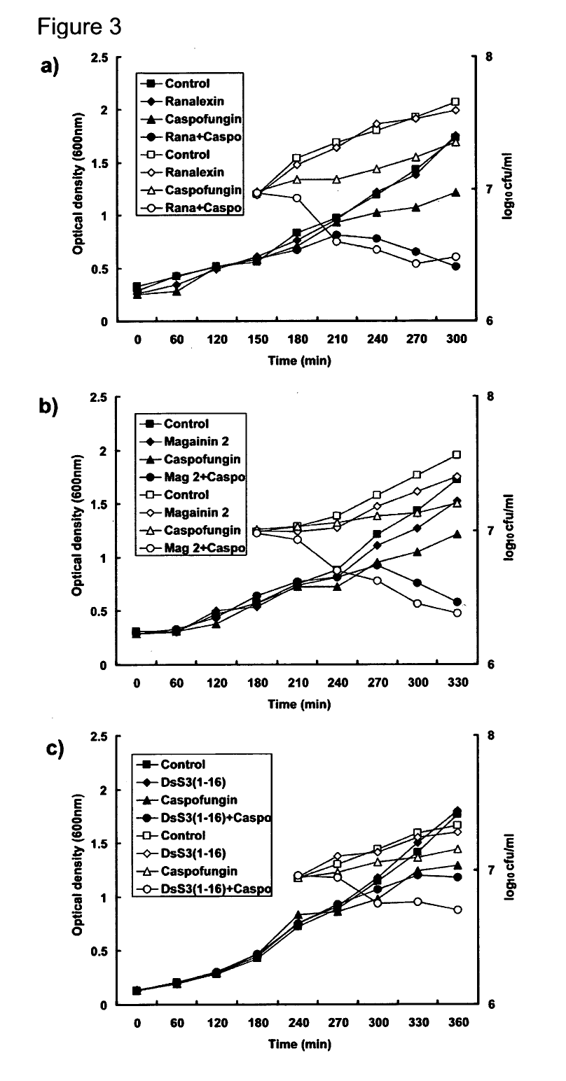

FIG. 3: shows the effect of caspofungin alone, peptide alone and a combination of both peptide and caspofungin on growth of a liquid culture of yeast measured by optical density change (OD600) and viable counts (% cell viability). Viability data is shown with the black lines and symbols. Growth data is shown with the coloured lines and symbols. The viable count data in particular clearly shows that combination of caspofungin with a peptide results in greatly enhanced killing of the yeast when compared to the effect of the individual antifungals alone.

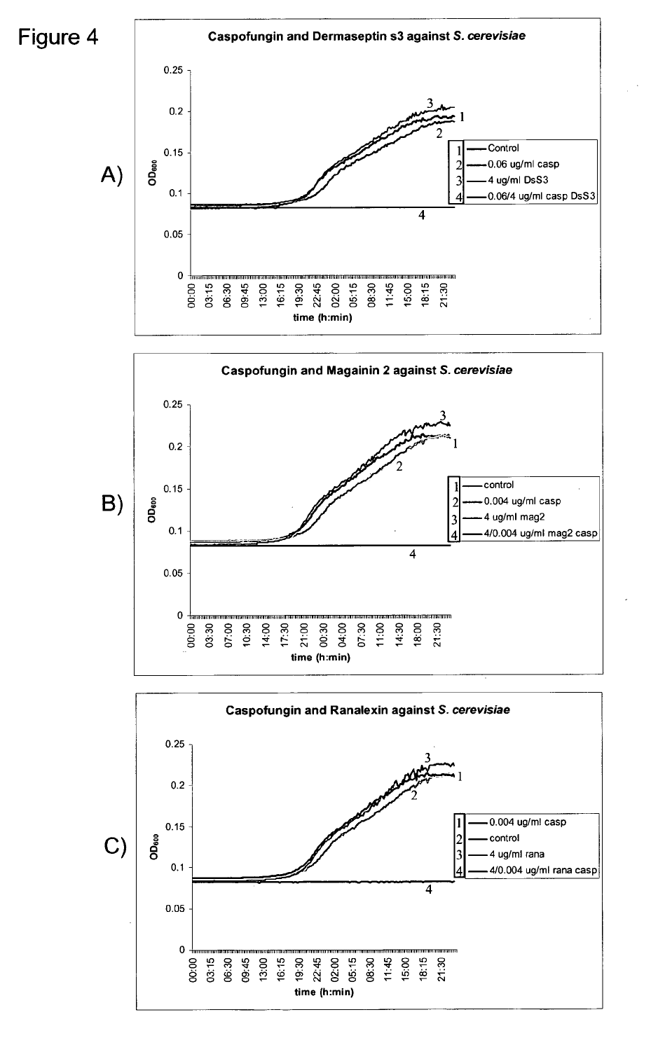

FIG. 4: shows individual yeast growth curves measured by increase in optical density (OD600) over a period of 24 h in the wells of a microtitre plate. The lines represent: Blue—untreated yeast culture alone; Yellow—yeast culture in the presence of a subinhibitory concentration of caspofungin alone; Green—yeast culture in the presence of a subinhibitory concentration of an antimicrobial peptide alone; Red—culture in the presence of a combination of sub-inhibitory caspofungin with sub-inhibitory peptide. In the case of each peptide (A: dermaseptin S3(1-16); B: magainin 2; C: ranalexin), combination with caspofungin results in the complete abolition of yeast growth indicating potent synergistic inhibition.

### MATERIALS AND METHODS

### Yeast Strains and Growth Media

Yeast strains used in this work were Saccharomyces cerevisiae BY4741 (MATa his3Δ1 leu2Δ0 met15Δ0 ura3Δ0) (research Genetics); Candida albicans SC5314; Candida albicans CAI-4, a gift from Prof. Neil Gow, University of Aberdeen; and clinical isolates of C. albicans and C. glabrata provided by Dr. Cyril Lafong, Fife Area Laboratory, Victoria Hospital, Kirkcaldy. All strains were cultured in 100 ml flasks with Malt Extract Broth (MEB), pH 7 (1% glucose, 0.6% malt extract, 0.12% yeast extract) at 30° C. with shaking. For enumerating cell survival after exposure to antimicrobials yeasts were plated on YEPD agar (2% glucose, 2% agar, 1% bactopeptone, 1% yeast extract). Numbers of cells used in the various assays described below were calculated using an optical density at 600 nm (OD₆₀₀) versus viable cell numbers calibration curve that was generated for each yeast strain used.

### Antifungals

Magainin 2, ranalexin, dermaseptin S3(1-16), 6752 and GS14K4 were synthesized according to their published sequences by Peptide Research Ltd, Wickham, UK, to greater than 95% purity and verified by HPLC and mass spectrometry. Colistin sulfate and nisin were obtained from Sigma. Polymyxin B was obtained from Fluka. Peptides were solubilised in d.H₂O at stock concentrations of 50 mg ml⁻¹. Caspofungin (Merck) and anidulafungn (Pfizer) was diluted into 1 mg ml⁻¹ aliquots with sterile d.H₂O.

### Checkerboard Assay of Yeast Growth Inhibition

96-well micro-titre plate (Greiner Bio-one Ltd, Stonehouse, UK) wells were filled with 150 μl MEB or RPMI-1640, 2% MOPS (Sigma), pH 7 and concentrations of either peptides or caspofungin alone, or combinations of peptides with caspofungin. Approximately 500 cells from mid-exponential phase cultures of the yeast strains described above were then added to each well. Plates were incubated at 30° C. for 48 hours and scanned as described above.

### Disc Diffusion Assay of Yeast Growth Inhibition

Sterile filter paper discs (6 mm, Aa, Whatman) were impregnated with either, peptides or caspofungin alone, or peptides and caspofungin together, and left in sterile Petri dishes overnight at room temperature to dry. Following this, 100 μl of mid-exponential phase culture of yeast was spread onto the surface of MEB, pH 7 plus 2% agarose plates and left to dry for 1 h at room temperature. MEB plates were made with 2% agarose rather than agar because the agar inhibited the action of the antimicrobial peptides. Discs containing the various antifungal combinations were then applied to the surface and the plates were incubated at 30° C. for 48 h. Images showing the zones of inhibition were obtained by scanning the plates using an ImageScanner (GE Healthcare UK Ltd, Chalfont St Giles, UK) with ImageMaster Labscan v 3.00 software (GE Healthcare UK Ltd). Images were annotated using Microsoft PhotoDraw.

### Liquid Culture Assay of Yeast Growth Inhibition

Sterile MEB, pH 7 was added (300 μl) to each well of 48-well microtitre plate (Greiner Bio-one Ltd, Stonehouse, UK) followed by appropriate concentrations of each peptide alone, caspofungin alone and various combinations of peptides with caspofungin. The wells were then inoculated with mid-exponential phase yeast cultures to give starting cell numbers of 1000 cells per well. The plates were incubated at 30° C., with shaking, in an automated microplate spectrophotometer (Bio-tek Instruments Inc., Winooski, Vt., USA) for 48 h. OD₆₀₀ readings were taken every 15 minutes. Data was analysed in Microsoft Excel.

### Assay of Yeast Cell Viability

Yeast starter cultures were incubated overnight in MEB, pH 7 at 30° C. with shaking. ml of yeast culture was diluted in 19 ml of sterile MEB, pH 7 to give starting cell numbers of approximately 1.0×10⁶ cells ml⁻¹. The fresh cultures were then incubated at 30° C. with shaking and OD₆₀₀ readings were taken every 60 min until an optical density of 0.3 (mid-exponential) was reached. Cultures were then exposed to appropriate concentrations of peptide with or without caspofungin. Culture viability was measured every 30 min by serial dilution and plating on YEPD agar plates. Plates were incubated at 30° C. for 48 h prior to counting.

### Results

### Table 1

Fractional inhibitory concentration (FIC) indices for combinations of caspofungin or anidulafungin with various antimicrobial peptides calculated from chequerboard assays of visible growth inhibition of three Candida strains tested in RPMI-1640. IC values highlighted in grey indicate combinations where no synergy was observed. Fractional Inhibitory Concentration (FIC)=(A+B)+(B+A);
A B
   where <0.5 synergy, >4 antagonism and 0.5 to 4 no interaction.
   (Odds, F. C. (2003) Synergy, antagonism, and what the chequerboard puts between them. J. Antimic. Chemother.)
   N/A—not tested.

| | Caspofungin | | | Anidulafungin | | |
|---|---|---|---|---|---|---|
| | | C. albicans | C. glabrata | | C. albicans | C. glabrata |
| | C. albicans | (Hospital | (Hospital | C. albicans | (Hospital | (Hospital |
| Peptide | SC5314 | isolate) | isolate) | SC5314 | isolate) | isolate) |
| DsS3(1-16) | 0.164 | 0.164 | 0.282 | 0.107 | 0.111 | 0.287 |
| Ranalexin | 0.221 | 0.164 | 0.186 | 0.106 | 0.111 | 0.443 |
| Magainin 2 | 0.144 | 0.135 | 0.278 | 0.095 | 0.087 | 0.376 |
| 6752 | 0.150 | 0.289 | 0.347 | 0.773 | 0.563 | 0.737 |
| GS14K4 | 0.267 | 0.139 | 0.269 | 0.142 | 0.287 | 0.341 |
| Polymyxin B | 0.310 | N/A | N/A | 0.590 | N/A | N/A |
| Colistin sulfate | 0.249 | N/A | N/A | 0.230 | N/A | N/A |
| Nisin | 0.315 | N/A | N/A | 0.491 | N/A | N/A |

1. A composition comprising an echinocandin and an antimicrobial peptide, for use in treating a fungal infection.

2. (canceled)

3. A method of treating a subject suffering from a fungal infection, said method comprising the steps of treating the subject with a therapeutically effective amount of an echinocandin and an antimicrobial peptide.

4. The composition, or method of claims 1 or 3, wherein the echinocandin is a lipopeptide which inhibits the synthesis of (1,3)-β-D-glucan.

5. The composition, or method of claims 1 or 3, wherein the echinocandin is selected from the group consisting of Echinocandin B, Cilofungin, Caspofungin (1-[(4R,5S)-5-[(2-aminoethyl)amino]-N2-(10,12-dimethyl-1-oxotetradecyl)-4-hydroxy-L-ornithine]-5-[(3R)-3-hydroxy-L-ornithine]pneumocandin B0), Micafungin and/or Anidulafungin.

6. The composition, or method of claims 1 or 3, wherein the fungal infection is a cutaneous, subcutaneous and/or systemic mycoses.

7. The composition, or method of claims 1 or 3, wherein the fungal infection is caused by one or more fungal species, selected from the group consisting of Epidermophyton, Microsporum, Trichophyton, Actinomadura, Cladosporium, Madurella, Phialophora, Sporothrix, Blastomyces, Coccidioides, Histoplasma, Paracoccidioides, Absidia, Candida, Aspergillus, Crytopcoccus, Pneumocystis, Rizomucor and Rhizopus.

8. The composition, or method of claims 1 or 3, wherein the antimicrobial peptide comprises 5-50 amino acids.

9. The composition, or method of claims 1 or 3, wherein the antimicrobial peptide is a linear, α-helical antimicrobial peptide having an amphipathic structure.

10. The composition, or method according to claims 1 or 3, wherein the antimicrobial peptide is a cationic peptide.

11. The composition, or method according to claims 1 or 3, wherein the antimicrobial peptide is a lipopeptide.

12. The composition, or method according to claims 1 or 3, wherein the antimicrobial peptide is a lantibiotic.

13. The compound, or method according to claims 1 or 3, wherein the antimicrobial peptide is one or more selected from the group consisting of ranalexin, dermaseptin S3(1-16), those of the magainin family such as, for example, magainin 2, 6752, GS14K4, Polymyxin B, Colistin sulfate (polymyxin E) and Nisin.

14. A formulation comprising one or more echinocandin compound(s) selected from the group consisting of Echinocandin B, Cilofungin, Caspofungin (1-[(4R,5S)-5-[(2-aminoethyl)amino]-N2-(10,12-dimethyl-1-oxotetradecyl)-4-hydroxy-L-ornithine]-5-[(3R)-3-hydroxy-L-ornithine]pneumocandin B₀), Micafungin and/or Anidulafungin. and one or more antimicrobial peptide(s) selected from the group consisting of:
(i) ranalexin;
(ii) magainin 2;
(iii) dermaseptin S3(1-16);
(iv) 6752;
(v) GS14K4;
(vi) Polymyxin B;
(vii) Colistin sulfate; and
(viii) Nisin for use in treating a fungal infection.

15. A pharmaceutical composition comprising an echinocandin and an antimicrobial peptide for use in treating a fungal infection, in association with a pharmaceutically acceptable excipient, carrier or diluent.

16. The pharmaceutical composition of claim 15 formulated for oral, parenteral or topical administration.

17. The composition, or method of claims 1 or 3, wherein the echinocandin compound and antimicrobial peptide is intended to be administered topically and/or transdermally.

18. The pharmaceutical composition according to claim 15 comprising one or more echinocandin compound(s) selected from the group consisting of Echinocandin B, Cilofungin, Caspofungin (1-[(4R,5S)-5-[(2-aminoethyl)amino]-N2-(10,12-dimethyl-1-oxotetradecyl)-4-hydroxy-L-ornithine]-5-[(3R)-3-hydroxy-L-ornithine]pneumocandin B₀), Micafungin and/or Anidulafungin. and one or more antimicrobial peptide(s) selected from the group consisting of:
(i) ranalexin;
(ii) magainin 2;
(iii) dermaseptin S3(1-16);
(iv) 6752;
(v) GS14K4;
(vi) Polymyxin B;
(vii) Colistin sulfate; and
(viii) Nisin
for use in treating a fungal infection.
